(19) United States
(12) Patent Application Publication
(10) Pub. No.: US 2022/0280665 A1
(43) Pub. Date: Sep. 8, 2022

(54) STERILIZATION DEVICE AND A METHOD THEREOF

(71) Applicant: Susan L. Quan, Morgan Hill, CA (US)

(72) Inventors: Susan L. Quan, Morgan Hill, CA (US); Rajiv Gupta, Saratoga, CA (US)

(73) Assignee: Susan L. Quan

(21) Appl. No.: 17/189,338

(22) Filed: Mar. 2, 2021

(51) Int. Cl. A61L 2/10; A61L 2/26

(52) U.S. Cl. CPC A61L 2/10; A61L 2/26; A61L 2202/122; A61L 2202/182; A61L 2202/24

(57) ABSTRACT

A sterilization device is disclosed. The device includes a housing that includes a sterilization conveyor chamber. The sterilization conveyor chamber includes a sterilization conveyor system and a tray placed near the sterilization conveyor system. The tray includes multiple compartments to collect objects. The sterilization conveyor chamber includes an UVC-LED source to sterilize the objects when exposed to UVC light. The sterilization conveyor chamber includes an outlet to receive sterilized objects one at a time. The housing includes a packaging chamber coupled to the sterilization conveyor system via the outlet. The packaging chamber envelopes and seals the sterile objects with a preloaded spool of packaging material. The housing also includes an object retrieval chamber comprising a drawer to store and protect the sterilized objects by moving the sterilized objects.

### FIELD OF INVENTION

Embodiments of the present disclosure relate to medical devices, and more specifically to a sterilization device for sterilization of objects and a method thereof.

### BACKGROUND

New microbiologic pathogens, namely viruses, are being discovered in various parts of the world that easily transfer disease from animals to humans, and then from humans to humans. Travel, congregate gatherings, and aerosolization of infectious materials can spread viral infection to entire populations at rapid rates leading to local and global pandemics. The current SARS-Cov2 virus is only one of many viruses in this family of pathogens that has rendered great health and economic burdens on entire populations and economies at a time. Viruses are carried and spread via personal contact and respiratory inhalation. To neutralize this threat in an effective and efficient manner, there is great need to develop technologies that break this chain of pathogenicity through rapid and powerful sterilization vehicles. Sterilization refers to any process that removes, kills, or deactivates all forms of life (in particular, referring to microorganisms such as fungi, bacteria, viruses, spores, unicellular eukaryotic organisms such as Plasmodium, or the like) and other biological agents like prions present on a specific surface, object, or fluid, for example food or biological culture media. Sterilization can be achieved through various means, including heat, chemicals, irradiation, high pressure, and filtration.

Respiratory pathogens require air management to limit aerosol inhalation. This is accomplished by wearing facial coverings, or face masks, that limit the aerosolization of viral particles. The current marketplace for sterilizing face masks, personal belongings, supplies, and instruments is rather self-limiting. Healthcare facilities that require instrument sterilization achieve this via chemical, steam, and pressure vessels that require structural plumbing in order to operate these vessels. However, the sterilization of facial coverings is haphazard, unreliable, and not standard practice. Attempts to sterilize certain kind of masks fall short on performance and reliability due to many factors. Many anti-virus masks among the prior art are mostly disposable. Additionally, many attempts to sterilize face masks against such anti-virus masks or other belongings do not withstand the high temperature, high pressure, and ultraviolet rays during various types of sterilization processes. On the other hand, if the temperature, pressure, or ultraviolet lights are used in low amounts, the anti-virus masks or the belongings do not effectively kill the persistent and microscopic pathogens. Current “retail” ultraviolet (UV) sterilizers in the marketplace do not pass the rigors of scientific testing to account for shadowing, reliability and efficiency.

Currently, conventional marketplace sterilizers require complex plumbing to operate, which limits the type of settings that can achieve sterilization of instruments. Some conventional sterilization devices use ultraviolet lamps as a sterilization means. However, such ultraviolet lamps result in high heat dissipation which leads to less efficient sterilization of masks and belongings and can cause inconvenience to the user. Hence, there is a need for an improved sterilization device to address the aforementioned issues.

### BRIEF DESCRIPTION

In accordance with one embodiment of the present disclosure, a sterilization device is provided. The device includes a housing. The housing includes a sterilization conveyor chamber. The sterilization conveyor chamber includes a motorized conveyor system placed at a bottom surface of the sterilization conveyor chamber. The sterilization conveyor chamber includes a tray placed on a first surface of the sterilization conveyor system, where the tray includes a plurality of compartments configured to collect an object from a user. The sterilization conveyor chamber further includes a UVC-LED source placed in the corresponding plurality of compartments. The UVC-LED source is configured to sterilize the object when exposed to UVC light. The sterilization conveyor chamber further includes an outlet located within the sterilization conveyor chamber, where the outlet is configured to receive a sterilized object one at a time shuttled by a conveyor system. The housing also includes a packaging chamber coupled to the conveyor system via the outlet. The packaging chamber is configured to envelope and seal the sterilized object received by the outlet with a preloaded spool of an environmental-friendly packaging material. The housing further includes an object retrieval chamber comprising a drawer configured to store and protect the sterilized object sealed by the packaging chamber by moving the sterilized object using the sterilization conveyor system to obtain a sterile package. This device will render medical-grade sterilization in a hands-free manner, thereby reducing the risk of operator exposure to contaminants and the need for clinician-controlled operation.

In accordance with another embodiment of the present disclosure, a method to assemble a sterilization device is provided. The method includes providing a housing comprising a sterilization conveyor chamber. The method also includes placing a sterilization conveyor system at a bottom surface of the sterilization conveyor chamber. The method further includes placing a tray on a first surface of the sterilization conveyor system, where the tray includes a plurality of compartments configured to collect an object from a user. The method further includes locating a UVC-LED source in the corresponding plurality of compartments, where the UVC-LED source is configured to sterilize the object when exposed to UVC light. The method further includes receiving a sterilized object via an outlet located within the sterilization conveyor chamber one at a time shuttled by the sterilization conveyor system. The method further includes coupling a packaging chamber to the sterilization conveyor system via the outlet, where the packaging chamber is configured to envelope and seal the sterilized object received by the outlet with a preloaded spool of an environmental-friendly packaging material. The method further includes providing an object retrieval chamber comprising a drawer configured to store and protect the sterilized object sealed by the packaging chamber by moving the sterilized object using the sterilization conveyor system to obtain a sterile package.

In yet another embodiment of the present disclosure, a mask sterilization device is provided. The device includes a housing including a sterilization conveyor chamber. The sterilization conveyor chamber includes a sterilization conveyor system placed at a bottom surface of the sterilization conveyor chamber. The sterilization conveyor chamber includes a tray placed on a first surface of the sterilization conveyor system, where the tray includes a plurality of compartments configured to collect a mask from a user. The sterilization conveyor chamber includes a UVC-LED source placed in the corresponding plurality of compartments, where the UVC-LED source is configured to sterilize the mask when exposed to UVC light. The sterilization conveyor chamber includes an outlet located within the sterilization conveyor chamber, where the outlet is configured to receive a sterilized mask one at a time shuttled by the sterilization conveyor system. The housing also includes a packaging chamber coupled to the sterilization conveyor system via the outlet, where the packaging chamber is configured to envelope and seal the sterilized mask received by the outlet with a preloaded spool of an environmental-friendly packaging material. The housing further includes an object retrieval chamber including a drawer configured to store and protect the sterilized mask sealed by the packaging chamber by moving sterilized mask using the sterilization conveyor system to obtain a sterile package.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation of an isometric view of a sterilization device in accordance with an embodiment of the present disclosure;

FIG. 2 is a schematic representation of internal view of the sterilization device of FIG. 1 in accordance with an embodiment of the present disclosure;

FIG. 3 is a schematic representation of one embodiment of sterilization device of FIG. 1 in accordance with an embodiment of the present disclosure;

FIG. 4 is a schematic representation of another embodiment of the sterilization device of FIG. 1, depicting operation of the sterilization device in accordance with an embodiment of the present disclosure; and

FIG. 5 is a flow chart representing the steps involved in a method to assemble the sterilization device in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but includes other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a sterilization device and a method thereof. The device includes a housing. The housing includes a sterilization conveyor chamber. The sterilization conveyor chamber includes a sterilization conveyor system placed at a bottom surface of the sterilization conveyor chamber. The sterilization conveyor chamber includes a tray placed on a first surface of the sterilization conveyor system, where the tray includes a plurality of compartments configured to collect an object from a user. The sterilization conveyor chamber further includes a UVC-LED source placed in the corresponding plurality of compartments. The UVC-LED source is configured to sterilize the object when exposed to UVC light. The sterilization conveyor chamber further includes an outlet located within the sterilization conveyor chamber, where the outlet is configured to receive a sterilized object one at a time shuttled by the sterilization conveyor system. The housing also includes a packaging chamber coupled to the sterilization conveyor system via the outlet. The packaging chamber is configured to envelope and seal the sterilized object received by the outlet with a preloaded spool of an environmental-friendly packaging material. The housing further includes an object retrieval chamber comprises a drawer configured to store and protect the sterilized object sealed by the packaging chamber by moving the sterilized object using the sterilization conveyor system to obtain a sterile package.

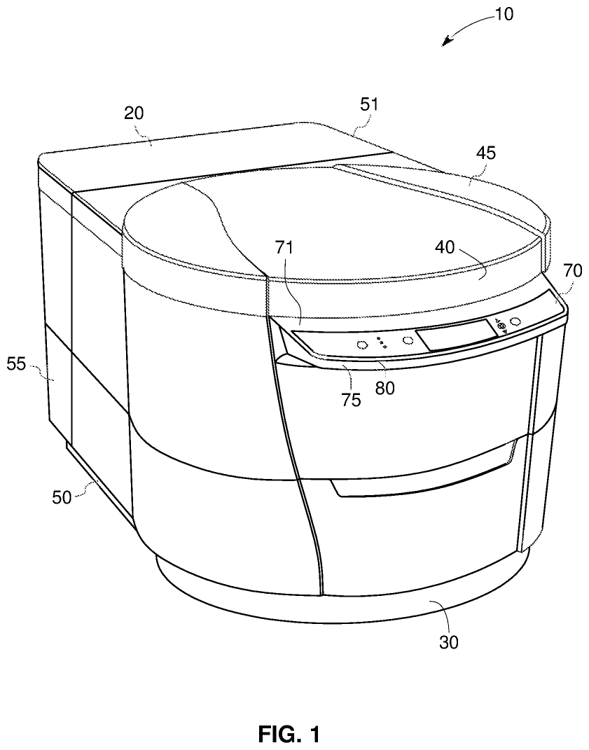

FIG. 1 is a schematic representation of an isometric view of a sterilization device 10 in accordance with an embodiment of the present disclosure. The sterilization device 10 includes a housing 20 having a base 30 and a lid 40. In one embodiment, the housing 20 includes a first side 45 constitutes the top wall of the housing 20. In such an embodiment, the housing 20 includes a second side 50 that constitutes the base 30 of the housing 20. In a specific embodiment, the housing 20 includes a first sidewall 55 that acts as a vertical wall that connects a top side 45 to a bottom side 50. The housing 20 includes a second sidewall 51 that is on the opposite of the first sidewall 55. The second sidewall 51 connects the first side 45 to the second side 50. In one embodiment, the housing 20 is composed of a material including plastic. In such an embodiment, the housing 20 includes a combination of rectangular and circular-shaped boxes. In some embodiments, the housing 20 includes an operating panel 70 positioned on an outside surface 71 of the housing 20. The operating panel 70 includes a power button 75 and a UV button 80 which are configured to enable the user to operate the sterilization device 10. The power button 75 is used to turn ON the operation of the sterilization device 10 and the UV button is configured to turn ON the UVC light inside the plurality of compartments to sterilize the object.

As disclosed herein, the present embodiment has the advantage of ease of use for the operator. Sterilization device 10 requires only an AC plug, and does not require the plumbing required to provide steam, pressure, etc. as current marketplace medical-grade sterilization vessels. This will be significant in organizations and locations that would not normally have the ability to achieve sterilization under current means. This includes, but are not limited to, rural locales and other non-medical facilities such as airplanes, corporations, manufacturing facilities, etc. As disclosed herein, this embodiment also potentially can reduce or eliminate the use of current certain sterilizers.

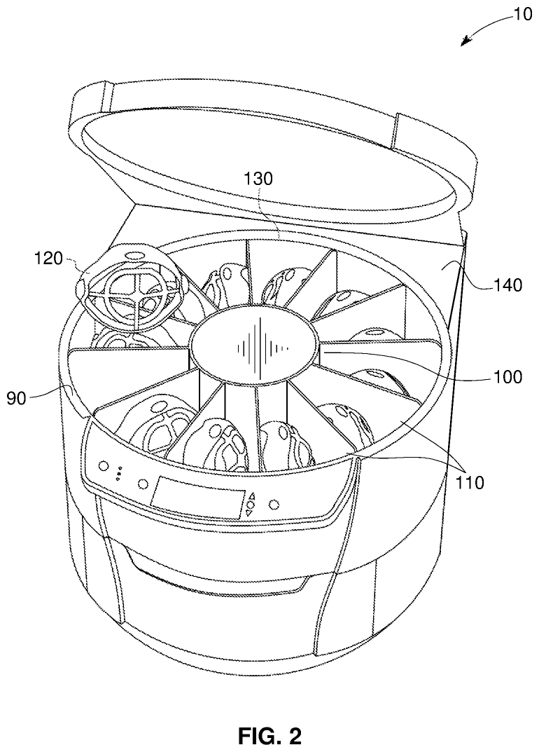

FIG. 2 is a schematic representation of an internal view of the sterilization device 10 of FIG. 1 in accordance with an embodiment of the present disclosure. The housing 20 includes a sterilization conveyor chamber 90. According to some embodiments, the sterilization device 10 includes a sterilization conveyor system (not shown in FIG. 2). According to some embodiments, the sterilization conveyor system is placed at bottom surface of the sterilization conveyor chamber. In yet other embodiments, the sterilization conveyor system is comprised of a robotic appendage or a belt system to transport items to and from the sterilization conveyor chamber 90. As used herein, the sterilization conveyor system includes of a drive roller powered by a drive motor with a tension roller at the opposite end moving an endless loop of the conveyor belt which rotates around them. The belt is supported by a “skid” base or a roller base. The belt is returned on a series of return rollers. In one embodiment, the sterilization conveyor systems are typically feature materials including rubber or a fabric such as nylon, polyester, neoprene, or nitrile. The belt consists of one or more layers of material. It is common for belts to have three layers such as a top cover, a carcass and a bottom cover. The purpose of the carcass is to provide linear strength and shape. The carcass is often a woven or metal fabric having a warp and weft. The warp refers to longitudinal cords whose characteristics of resistance and elasticity define the running properties of the belt. The weft represents the whole set of transversal cables allowing to the belt specific resistance against cuts, tears and impacts and at the same time high flexibility.

Furthermore, the sterilization conveyor chamber 90 also includes a tray 100 which is placed on top of the sterilization conveyor system. The tray 100 includes a plurality of compartments 110 which is configured to collect an object 120 from a user. In one embodiment, the object 120 includes at least one of a mask, keys, PPE kit, currency or the like or a combination thereof. Subsequently, the sterilization conveyor chamber 90 further includes an ultraviolet-C light emitting diode (UVC-LED) source (not shown in FIG. 2) which are placed in the corresponding plurality of compartments. In one embodiment, the UVC-LED source is an integrated circuit or a printed circuit board. The plurality of UVC-LED source is configured to sterilize the object when exposed to UVC light. In one embodiment, the sterilization of the object includes a turn-around-time is less than 5 minutes. The turn-around-time is a time required to complete steps of insertion, sterilization and removal of the sterilized object. As used herein, the UVC region of the UV spectral range refers to wavelengths between 100 nm to 280 nm. The UVC-LED source produce photons which are directed at viruses, bacteria and other pathogens present on the object placed in the corresponding plurality of compartments. The photons penetrate cells and damage the nucleic acid, rendering them incapable of reproduction, or microbiologically inactive during sterilization process.

Moreover, the sterilization conveyor chamber 90 includes an outlet 130 which is located within the sterilization conveyor chamber. The outlet 130 is configured to receive a sterilized object 120 one at a time shuttled by the sterilization conveyor system. Further, the housing 20 includes a packaging chamber 140 which is coupled to the sterilization conveyor system via the outlet. The packaging chamber 140 is configured to envelope and seal the sterilized objects received by the outlet 130 with a preloaded spool of an environmental-friendly packaging material. In one embodiment, the preloaded spool of the environmental-friendly packaging material includes at least one of medical-grade paper, polymer and high-density polyethylene (HDPE).

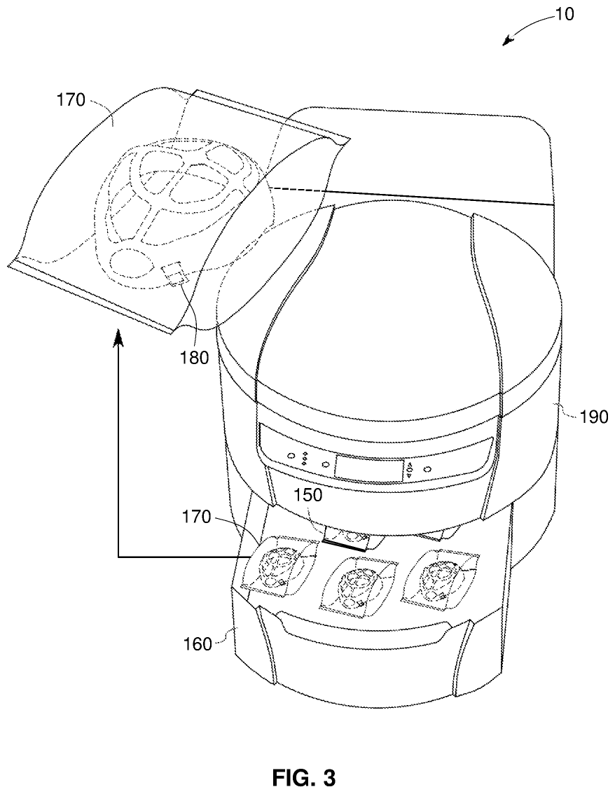

FIG. 3 is a schematic representation of one embodiment of sterilization device 10 of FIG. 1 in accordance with an embodiment of the present disclosure. The housing 20 includes an object retrieval chamber 150 including a drawer 160 configured to store and protect the sterilized object sealed by the packaging chamber by moving the sterilized object using the sterilization conveyor system to obtain a sterile package 170. In one embodiment, the sterile package 170 comprises a sterility indicating marker 180. As used herein, the sterility indicating marker, such as spore strips and indicator tape, enable routine monitoring, qualification, and load monitoring of the sterilization process. The sterility indicating marker depicts whether the conditions during a sterilization cycle were adequate to achieve a defined level of microbial inactivation. In a specific embodiment, the sterility indicating marker is a mechanical marker, a chemical marker and a biological marker. The chemical indicating marker should be used inside every package to verify that the sterilizing agent has penetrated the package and reached the objects inside. If the internal chemical indicator is not visible from the outside of the package, an external indicator should also be used. Chemical indicators help to differentiate between processed and unprocessed objects, eliminating the possibility of using objects that have not been sterilized.

In one embodiment, the housing 20 includes a lid 40 having a locking mechanism to enable locking of the lid 40 during sterilization of the object. In such an embodiment, the lid 40 is coupled to one or more sensors (not shown in FIG. 3) configured to sense exposure of the UVC light. When the one or more sensors sense the exposure of UVC light during the sterilization process, such signal is provided to the locking mechanism of the lid 40 which locks the lid 40 during the whole sterilization process to safeguard from the exposure of harmful UVC on the user. In some embodiments, the housing 20 includes a chamber controller which is coupled to the sterilization conveyor system, the UVC chips, the packaging chamber, the object retrieval chamber and one or more sensors. In such an embodiment, the chamber controller is configured to receive power supply from a power distribution unit and control operation of the sterilization conveyor system, the UVC-LED chips, the packaging chamber, the object retrieval chamber and one or more sensors. In one embodiment, the housing 20 includes an LED indicator 190 configured to glow during operation of the UVC-LED source, the packaging chamber and the object retrieval chamber.

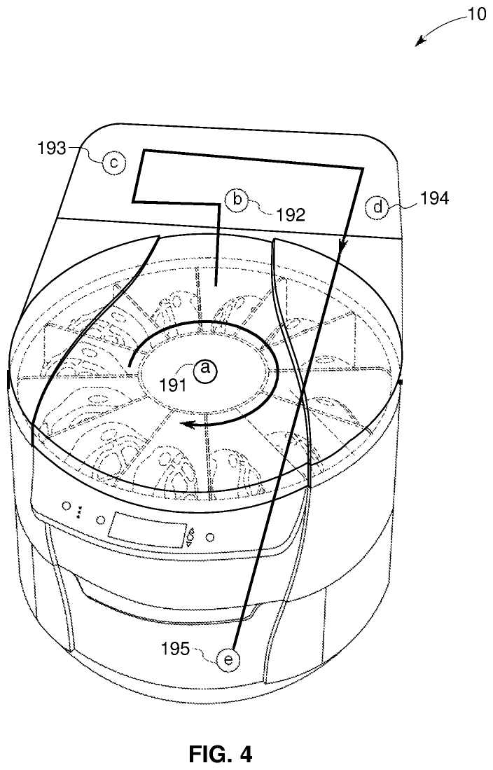

FIG. 4 is a schematic representation of another embodiment of the sterilization device 10 of FIG. 1, depicting operation of the sterilization device 10 in accordance with an embodiment of the present disclosure. Considering an example where the sterilization device 10 is used to sterilize one or more masks. The one or more masks are placed in corresponding compartments. At step 191, the sterilization conveyor system rotates to position next mask for processing. At step 192, the mask at rear of sterilization conveyor system is shuttled into UV chamber at rear of device. At step 193, the sterilization marker is applied in the outlet and the one or more masks are sterilized with UV-C Light. At step 194, the one or more masks are shuttled to packaging station where it is heat sealed into bag which is of a preloaded spool of an environmental-friendly packaging material. At step 195, the sterilized and sealed masks are robotically placed into the drawer of the object retrieval chamber. During the whole sterilization process, a blue band which is an LED indicator, glows to indicate the sterilization cycle. The bagged mask with sterility indicating marker is collected from the drawer upon completion of the sterilization process.

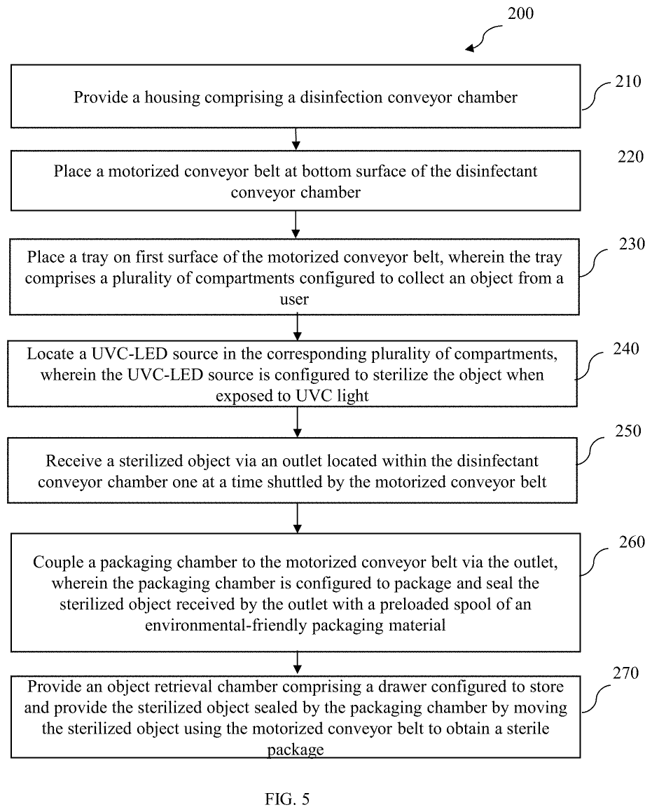

FIG. 5 is a flow chart representing the steps involved in a method 200 to assemble the sterilization device 10 in accordance with an embodiment of the present disclosure. The method 200 includes providing a housing, such as housing 20 of FIG. 1, comprising a sterilization conveyor chamber in step 210. In one embodiment, the housing 20 includes a lid 40 having a locking mechanism to enable locking of the lid 40 during sterilization of the object. In such an embodiment, the lid 40 is coupled to one or more sensors configured to sense exposure of the UVC light. In a specific embodiment, the housing 20 is composed of a material including plastic. In some embodiments, the housing 20 includes an operating panel in an outside surface. The operating panel includes a power button and a UV button configured to enable the user to operate the sterilization device 10. In one embodiment, the housing 20 includes the top side 45 constitutes the top wall, the bottom side 50 constitutes the base 30, the first sidewall 55 is a vertical wall connects the top side 45 to the bottom side 50 and the second sidewall 51 is on the opposite of the first sidewall 55 and the second sidewall 51 and connects the top side 45 to the bottom side 50.

The method 200 also includes placing a sterilization conveyor system at bottom surface of the sterilization conveyor chamber in step 220. The method 200 further includes placing a tray on top of the sterilization conveyor system. The tray includes a plurality of compartments configured to collect an object from a user in step 230. In one embodiment, the object includes at least one of masks, keys, PPE kit or the like or a combination thereof. The method further includes locating a UVC-LED source in the corresponding plurality of compartments. The UVC-LED source is configured to sterilize the object when exposed to UVC light in step 240. In a specific embodiment, the sterilization of the object comprises a turn-around-time of less than 5 minutes.

Additionally, the method 200 further includes receiving a plurality of sterile objects via an outlet located within the sterilization conveyor chamber one at a time shuttled by the sterilization conveyor system in step 250. The method 200 further includes coupling a packaging chamber to the sterilization conveyor system via the outlet. The packaging chamber is configured to envelope and seal the plurality of sterile objects received by the outlet with a preloaded spool of an environmental-friendly packaging material in step 260. In one embodiment, the preloaded spool of the environmental-friendly packaging material comprises at least one of medical-grade paper, polymer and high-density polyethylene (HDPE).

The method 200 further includes providing an object retrieval chamber comprising a drawer configured to store and protect the plurality of sterile objects sealed by the packaging chamber by dropping the plurality of sterile objects using the sterilization conveyor system to obtain a sterile package in step 270. In one embodiment, the housing 20 includes a chamber controller coupled to the sterilization conveyor system, the UVC-LED source, the packaging chamber, the object retrieval chamber and one or more sensors. In such an embodiment, the chamber controller is configured to receive power supply from a power distribution unit and control operation of the sterilization conveyor system, the UVC-LED source, the packaging chamber, the object retrieval chamber and one or more sensors. In a specific embodiment, the housing 20 includes an LED indicator configured to glow during operation of the UVC-LED source, the packaging chamber and the object retrieval chamber.

Various embodiments of the sterilization device 10 as described above enables an UVC-LED object Sterilizer combines next-gen UVC-LED technology and cutting-edge robotics to deliver medical-grade performance sterilization of various objects such as face masks at a high throughput rate. The result is a completely sterilized, packaged and sealed objects delivered to wearers in a variety of clinical, corporate, manufacturing, services and retail settings. The device provides a no-touch process by allowing wearers to disrobe a worn mask and load onto compartments coupled to a sterilization conveyor system. The strategic placement of LED UVC source within the unit provides for complete sterilization of masks which then will be robotically moved to a packaging station within the sterilizer. Sterilization pouches will self-seal and be dropped into a drawer within the device, ready for dispensing when needed. This ensures the wearer a completely sterilized mask every time.

The device provides medical-grade PPE protection in a variety of settings. Fast throughput performance satisfies the demands of large-scale users in a broad range of industries. Cutting-edge robotics that provides for packaging and sealing of sterilized masks ensure that wearers will have a completely sterilized mask every time. Storage capacity allows for finished products to be ready for use on-demand. The ability to sterilize masks will result in less reliance on a fickle supply chain (expected to last in the foreseeable future). The ability to sterilize masks will result in less paper demand, waste and environmental insult, and greatly increase environment stewardship by organizations using the device. The device will provide reliable sterilization through rigorous performance testing and monitoring that retail units do not provide. The device will provide near-touchless operations, thereby minimizing cross-contamination risk.

In addition, the device provides a compact, self-contained and self-operating system. It is relatively easy adaptation to a number of environments will provide reliable PPE facial protection in environments that currently have none, and greatly improve others, namely hospitals. The device addresses barriers with cutting edge technology that minimizes infrastructure requirements and labor-intensive operations. Further, the design of the device will provide reliable performance outcomes in a controlled system.

The device may be utilized in various application areas such as hospitals, mid-size healthcare facilities, convalescent homes, dental offices, corporate offices, manufacturing lines, schools, institutions of higher education such as medical and dental schools, airplanes, massage providers, nail salons, restaurants, malls. The device will safely and reliably allow any business in which employees and customers congregate to operate with assurance that the highest level of PPE protection is provided. There is also retail potential, though this is not the primary target market. Additionally, the device would serve markets in which access to the supply chain would be greatly challenged such as remote locales, developing nations, or the like.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A sterilization device comprising:
a housing comprising:
a sterilization conveyor chamber, wherein the sterilization conveyor chamber comprises:
a sterilization conveyor system placed at a bottom surface of the sterilization conveyor chamber;
a tray placed on a first surface of the sterilization conveyor system, wherein the tray comprises a plurality of compartments configured to collect an object from a user;
a UVC-LED source placed in the corresponding plurality of compartments, wherein the UVC-LED source is configured to sterilize the object when exposed to UVC light;
an outlet located within the sterilization conveyor chamber, wherein the outlet is configured to receive a sterilized object one at a time shuttled by the sterilization conveyor system;
a packaging chamber coupled to the sterilization conveyor system via the outlet, wherein the packaging chamber is configured to envelope and seal the sterilized object received by the outlet with a preloaded spool of an environmental-friendly packaging material; and
an object retrieval chamber comprising a drawer configured to store and protect the sterilized object sealed by the packaging chamber by moving the sterilized object using the sterilization conveyor system to obtain a sterile package.

2. The device of claim 1, wherein the housing comprises a lid having a locking mechanism to enable locking of the lid during sterilization of the object.

3. The device of claim 3, wherein the lid is coupled to one or more sensors configured to sense presence of UVC light to ensure the device is not opened during sterilization of the object.

4. The device of claim 1, wherein the housing comprises a chamber controller coupled to the sterilization conveyor system, the UVC-LED source, the packaging chamber, the object retrieval chamber, and the one or more sensors.

5. The deice of claim 4, wherein the chamber controller is configured to receive power supplied from a power distribution unit to control operation of the sterilization conveyor system, the UVC-LED source, the packaging chamber, the object retrieval chamber, and the one or more sensors.

6. The device of claim 1, wherein sterilization of the object comprises a turn-around-time is less than 70 seconds, wherein the turn-around-time comprises time required to complete steps of insertion, sterilization and removal of the sterilized object.

7. The device of claim 1, wherein the housing comprises an LED indicator configured to glow during operation of the UVC-LED source, the packaging chamber, and the object retrieval chamber.

8. The device of claim 1, wherein the sterile package comprises a sterility indicating marker.

9. The device of claim 1, where the preloaded spool of the environmental-friendly packaging material comprises at least one of medical-grade paper, polymer, and high-density polyethylene (HDPE).

10. The device of claim 1, wherein the housing is composed of a material comprising plastic.

11. The device of claim 1, wherein the object comprises at least one of a mask, a set of keys, a PPE kit and currency.

12. The device of claim 1, wherein the housing comprises an operating panel positioned on an outside surface of the housing, wherein the operating panel comprises a power button and a UV button configured to enable the user to operate the sterilization device.

13. The device of claim 1, wherein the housing comprises:
a first side constitutes a top wall of the sterilization conveyor chamber;
a second side constitutes a base of the sterilization conveyor chamber;
a first sidewall is a vertical wall connects the first side to the second side; and
a second sidewall located opposite to the first sidewall and connects the first side to the second side.

14. A method comprising:
providing a housing comprising a sterilization conveyor chamber;
placing a sterilization conveyor system at a bottom surface of the sterilization conveyor chamber;
placing a tray on a first side of the sterilization conveyor system, wherein the tray comprises a plurality of compartments configured to collect an object from a user;
locating a UVC-LED source in the corresponding plurality of compartments, wherein the UVC-LED source is configured to sterilize the object when exposed to UVC light;
receiving a sterilized object via an outlet located within the sterilization conveyor chamber one at a time shuttled by the sterilization conveyor system;
coupling a packaging chamber to the sterilization conveyor system via the outlet, wherein the packaging chamber is configured to envelope and seal the sterilized object received by the outlet with a preloaded spool of an environmental-friendly packaging material; and
providing an object retrieval chamber comprising a drawer configured to store and protect the sterilized object sealed by the packaging chamber by moving the sterilized object using the sterilization conveyor system to obtain a sterile package.

15. The method of claim 14, wherein providing the housing comprises providing a lid having a locking mechanism to enable locking of the lid during sterilization of the object.

16. The method of claim 15, wherein providing the lid comprises providing one or more sensors coupled to the lid, wherein the one or more sensors are configured to sense presence of UVC light to ensure the device is not opened during sterilization of the object.

17. The method of claim 14, wherein providing the housing comprises providing an LED indicator configured to glow during operation of the UVC-LED source, the packaging chamber, and the object retrieval chamber.

18. The method of claim 14, wherein providing the housing comprises providing an operating panel positioned on an outside surface of the housing, wherein the operating panel comprises a power button and a UV button configured to enable the user to operate the sterilization device.

19. The method of claim 14, wherein providing the object retrieval chamber to obtain the sterile package comprises providing a sterility indicating marker.

20. A mask sterilization device comprising:
a housing comprising:
a sterilization conveyor chamber, wherein the sterilization conveyor chamber comprises:
a sterilization conveyor system placed at a bottom surface of the sterilization conveyor chamber;
a tray placed on a first surface of the sterilization conveyor system, wherein the tray comprises a plurality of compartments configured to collect a mask from a user;
a UVC-LED source placed in the corresponding plurality of compartments, wherein the UVC-LED source is configured to sterilize the mask when exposed to UVC light;
an outlet located within the sterilization conveyor chamber, wherein the outlet is configured to receive a sterilized mask one at a time shuttled by the sterilization conveyor system;
a packaging chamber coupled to the sterilization conveyor system via the outlet, wherein the packaging chamber is configured to envelope and seal the sterilized mask received by the outlet with a preloaded spool of an environmental-friendly packaging material; and
an object retrieval chamber comprising a drawer configured to store and protect the sterilized mask sealed by the packaging chamber by moving sterilized mask using the sterilization conveyor system to obtain a sterile package.
